(19) United States
(12) Patent Application Publication
(10) Pub. No.: US 2001/0036658 A1
(43) Pub. Date: Nov. 1, 2001

(54) Culture media for growing spirochetes

(76) Inventors: Steven E. Phillips, Ridgefield, CT (US); Hamid Moayad, Bedford, TX (US); Lida Mattman, Grosse Pointe, MI (US)

Correspondence Address: Toby H. Kusmer, McDERMOTT, WILL & EMERY, 28 State Street, Boston, MA, 02109, US

(21) Appl. No.: 09/728,252

(22) Filed: Dec. 1, 2000

Related U.S. Application Data
(63) Continuation-in-part of application No. 08/994,295, filed on Dec. 19, 1997.
(60) Provisional application No. 60/168,544, filed on Dec. 2, 1999.
(60) Provisional application No. 60/239,960, filed on Oct. 13, 2000.

(51) Int. Cl. C12N 1/20; C12Q 1/02

(52) U.S. Cl. 435/253.6; 435/29

(57) ABSTRACT

Culture media for use in the growth of mutant bacteria, particularly for the growth of sub-types of bacteria, generally known as cell wall deficient organisms, especially spirochetes. A method for reliably and reproducibly culturing various forms of cell wall deficient organisms from the blood of ill patients, and particularly for reliably and reproducibly culturing spirochetes from the blood and other body fluids of patients with chronic Lyme Disease or with multiple sclerosis.

### CLAIM OF PRIORITY

This application is a continuation-in-part of U.S. patent application Ser. No. 08/994,295, filed Dec. 19, 1997. This application also claims priority to U.S. provisional patent applications No. 60/168,544, filed Dec. 2, 1999, and 60/239,960, filed Oct. 13, 2000.

### FIELD OF THE INVENTION

The invention relates to culture media for use in the growth of bacteria having a particular form. More particularly, the invention relates to culture media which are suitable for the growth of subtypes of bacteria, generally known as cell wall deficient organisms, and has been found to be particularly useful for the growth of spirochetal organisms. The invention also relates to a method for reliably and reproducibly culturing various forms of cell wall deficient organisms from the blood of ill patients, and particularly for reliably and reproducibly culturing spirochetes from the blood of patients with chronic Lyme Disease and from the blood of patients with multiple sclerosis (MS).

### BACKGROUND OF THE INVENTION

Cell wall deficient organisms are mutant forms, which have been demonstrated as deriving from virtually every class of bacteria and fungus. Historically, the culture media which are available in the art to grow various non-cell wall deficient organisms have not been found to be suitable for the sustained growth of cell wall deficient organisms. Therefore, there exists a need for suitable culture media that are capable of sustaining the growth of a large variety of cell wall deficient organisms.

While numerous culture media are available in the art, all of which are to varying degrees effective as growth media for various other organisms, the art has been notably deficient in providing any culture medium which could be effectively utilized to grow any of the subtypes of bacteria classified as cell wall deficient organisms.

In the field of infectious disease, the gold standard for laboratory diagnosis is usually the recovery of the causative infectious agent from the infected host. Since this has been an extraordinarily rare event with chronic Lyme Disease, clarification of the nature of the illness, and proving its etiology as infectious, has been difficult.

Lyme Disease is a multi-system illness caused by infection with Borrelia burgdorferi. Its manifestations can be myriad. This, coupled with problems in current serologic assays, leads to frequent misdiagnosis at all stages of the illness. Some investigators believe that Lyme borreliosis is over diagnosed, while others maintain that it is under diagnosed. To further confuse matters, a significant percentage of patients with Lyme Disease relapse despite antibiotic therapy.

Chronic Lyme Disease is a controversial topic. Even after extended antibiotic treatment, persistent infection as the etiology for chronic Lyme Disease has been strongly suggested by the persistence of borrelial antigen, as demonstrated by polymerase chain reaction (PCR). However, these diagnostic tests are plagued by the absence of a gold standard. The gold standard for laboratory diagnosis in the field of infectious diseases has usually involved culture of the causative organism from the infected host. With Lyme Disease, attempts at this have been disheartening, if not total failures.

The organism responsible for Lyme Disease has occasionally been cultured from cases of treated late stage disease, but these have been primarily from cerebrospinal fluid and synovial fluid. Culture of the organism from blood has been a genuine rarity, and those that have successfully cultured have been primarily from cases of untreated, early disease.

Multiple sclerosis (MS) is an enigmatic demyelinating disorder recognized since 1884. Presently, it is generally believed to be an autoimmune illness. Genetic differences in susceptibility MS are well known and quite likely multifactorial, but have been shown to be at least partly due to variation in myelin composition. In this condition, it: is abundantly clear that sensitized T cells progressively destroy the patient's myelin. However, it is also abundantly clear that its epidemiology suggests that of an infectious disease.

Observations such as case clustering and correlation of disease prevalence with geographic latitude raise suspicion further. A preponderance of MS occurs in individuals who spent some portion of their formative years between the 38^{th} and 52^{nd} latitudes. There is also a well-documented increased incidence among doctors and nurses.

Various theories as to modes of transmission of postulated infectious agents have ranged from well waterborne, to household pets to mosquitoes. Another theory postulates that the organism comes from the patient's own oral flora, moving up to penetrate the central nervous system when the hard palate is at a relatively thin growth stage. Yet despite these various theories as to microbial trigger, no conclusive link has been found.

In the past, spirochetes have been associated with MS in three investigative approaches. In the first approach, animal inoculation of MS spinal fluid has resulted in spirochetosis in guinea pigs, hamsters, monkeys, and rabbits. When young rabbits were inoculated, paralysis and death resulted. Animal inoculation data were extended by a careful, intriguing experiment. Human lymphocytes from MS spinal fluid were injected into severe-combined immunodeficient (SCID) mice. These animals developed paralysis and ataxia. This result has two possible explanations. Either the T cells were able to overcome the species barrier and damaged the murine myelin; or possibly a clandestine growth stage of a pathogen was within the lymphocytes or concentrated on them as the spinal fluid was centrifuged. Interestingly, bacterial L-forms, i.e., cell wall deficient forms tend to circulate intracellularly in vivo.

A second approach has been to apply spinal fluid with and without centrifugation. Large numbers of spirochetes have been demonstrated in spinal fluids and autopsy specimens. Spirochetes from MS autopsy tissue have reacted with antibody recovered from the blood of MS patients' (for example, Gabriel Steiner's work; see, Steiner, 11(4) J Neuropathol. Exp. Neurol. 343 (1952)).

A third approach has been culture. Compared to inoculation and staining there have been far fewer attempts. Mainly, it is thought due to the fact that the medium that was historically used was quite prone to contamination, since it contained both non-autoclaved rabbit and human serum. Unfortunately, autoclaving of that particular medium lessens its utility considerably. The inventors' laboratory work has reached the same conclusion, however they have used a far superior medium in that it possesses superior growth characteristics and is uniformly free of contaminants.

The Applicants know of no prior art references which are directed to culture media which are specifically designed for or suitable for use in the culture from patients of cell wall deficient organisms such as, for example, the cell wall deficient spirochetes, i.e. the organisms that cause Lyme Disease and likely the ones that cause multiple sclerosis.

These and other objects of the invention will become apparent from the following more detailed discussion of the invention.

### SUMMARY OF THE INVENTION

The invention provides for culture media for use in the growth of bacteria having a particular form. More particularly, the invention provides for culture media that are suitable for the growth of sub-types of bacteria, generally known as cell wall deficient organisms. The invention also relates to a method for reliably and reproducibly culturing various forms of cell wall deficient organisms from the blood of ill patients, particularly for reliably and reproducibly culturing spirochetes, for example Borrelia, for example, B. burgdorferi, from the blood and body fluids of patients with chronic Lyme Disease or patients with multiple sclerosis.

In a preferred embodiment, the invention provides for a culture medium for the growth of cell wall deficient organisms, which has the following components:

proteose peptone

beef, infusion from

dextrose

sodium chloride

dipotassium phosphate

sodium thioglycollate

agar

bacto methylene blue

sucrose soluble starch

The compositional limitations and obvious advantages of the cultures provided for by the invention will be more clearly understood from the following description of the various embodiments.

### DETAILED DESCRIPTION OF THE INVENTION

The invention is directed to culture media for use in the growth of mutant bacteria having a particular form. In particular, the invention relates to a culture medium that is suitable for the growth of sub-types of bacteria, generally known as cell wall deficient organisms. The invention also relates to a method for reliably and reproducibly culturing various forms of cell wall deficient organisms from the blood of ill patients, particularly for reliably and reproducibly culturing spirochetes, such as Borrelia, including, B. burgdorferi, from the blood of patients with chronic Lyme Disease.

In studies undertaken to date, Applicants have been able to reliably and reproducibly isolate Borrelia burgdorferi from the blood and body fluids of patients with Lyme Disease who have been chronically symptomatic despite extensive antibiotic therapy. Applicants have also been able to isolate spirochetes from the blood and body fluids of patients with multiple sclerosis.

Therefore, it is concluded that the culture media taught and claimed in this application are useful alternatives to the available culture media which have to date been made available to the art in carrying out the growth of various types of sub type bacteria which fall within the classification of cell wall deficient organisms.

The following EXAMPLES are presented in order to more fully illustrate the preferred embodiments of the invention. These examples should in no way be construed as limiting the scope of the invention, as defined by the appended claims.

### EXAMPLE I

General MPM Culture Medium

Unless stated otherwise in connection with a particular example, the following methodology is employed in carrying out the tests reflected in the EXAMPLES:

MPM Medium:

To one liter of water is added; proteose peptone 20 g, beef infusion from 1000 g, dextrose 10 g, sodium chloride 10 g, dipotassium phosphate 4 g, sodium thiogycollate 1 g, purified agar 1 g, bacto methylene blue 0.004 g, sucrose 100 g, soluble starch 5 g. The MPM medium is autoclaved for 13-15 minutes at 120 degrees C. (The source of the water is not critical. For example, Detroit tap water and distilled water have been successfully used for preparing this medium). For the medium to be used in tube or slide culture, it must be stored refrigerated for 24 hours prior to final preparation.

For the MPM medium to be used in tubes, 10 ml of medium are boiled to dissolve the agar just before use and the following are added to each tube: 1 ml separately autoclaved yeast extract (e.g., Difco 0127) from a 10% solution to give a (preferably) final concentration of 1% and 1 ml of sterile 10% NaHCO₃. Alternatively, the final concentration of yeast extract can be in the range 0.5% to 1.5%. Since yeast extract may contain heat resistant bacilli, it is separately autoclaved for 30 minutes and batch tested for sterility. The inoculum is 0.1 ml of blood to 4 ml of medium in a slender screw top tube. Incubation is at 30 degrees Celsius under normal atmospheric conditions.

For the MPM medium to be used in slide culture, it is sterilized in 30 ml amounts in screw top tubes. Just before use, the medium is boiled to melt the agar and, when cool, but not solidified, the following are added: 3 ml of separately autoclaved 10% yeast extract and 10 ml of sterile 10% NaHCO₃. (Alternatively, the final concentration of yeast extract can be in the range 0.5% to 1.5%.) The broth is then poured aseptically into a sterile plastic Coplin jar. Slides are smeared with the patient's chosen bodily fluid. The slides must be specialized so as not to require fixative (e.g., Fisher Superfrost Plus). The smears are dried in an aseptic environment before being placed in the Coplin jar. Once inside, the lid is tightly closed and incubation is at 30 degrees C. Placement in the Coplin jar allows for varying gradations of oxygen tension. With this technique, growth of B. burgdorferi can often be seen within 20 hours, appearing as a band near the upper end of the smear.

For the MPM medium to be used for blood agar plates, the MPM broth medium is supplemented in 20 g of purified agar and modified by adding a total of 16 g. 6 ml of sheep's blood is added to achieve a total concentration of 6% sheep's blood as soon as the medium is removed from the autoclave, resulting in “chocolate agar.” At this point, separately autoclaved yeast extract is added to give a final concentration of 0.5% to 1.5%, preferably 1%. The medium is then poured into sterile plastic Petrie dishes and stored under refrigeration for 24 hours once solidified. The chocolate agar plates can then be incubated in a sealed jar with CO₂ supplied by candle extinction and partial anaerobiosis resulting from BBL Gas Paks.

### EXAMPLE II

Preparation of Basic MPM Culture Medium

Using the foregoing methodology, a basic MPM culture medium is prepared by adding to one liter of water the following components in the amounts indicated:

| Component | Amount |
|---|---|
| proteose peptone | 20 g |
| beef, infusion from | 1000 g |
| dextrose | 10 g |
| sodium chloride | 10 g |
| dipotassium phosphate | 4 g |
| sodium thioglycollate | 1 g |
| agar | 1 g |
| bacto methylene blue | .004 g |
| sucrose | 100 g |
| soluble starch | 5 g |

### EXAMPLE III

Preparation of MPM Culture Medium in Tubes

Tubes containing 10 ml of Basic MPM Culture Medium are boiled to dissolve the agar just before use. The following are added to each tube:

1 ml 10% sterile yeast extract (Difco 0127)*

1 ml sterile—10% NaHCO₃ *Yeast extract contains many heat resistant bacilli, hence it must first be autoclaved for 30 minutes and tested for sterility. Alternatively, the final concentration of yeast extract can be in the range 0.5% to 1.5%.

### EXAMPLE IV

Preparation of Basic MPM Culture Medium for Slide Culture

The medium is sterilized in 30 ml amounts in screw cap tubes. Just before use, the medium is boiled to melt the agar and, when cooled, but not solidified, the following are aseptically added to each tube:

3 ml 10% sterile yeast extract (Difco 0127)

10 ml sterile 10% NaHCO₃

Alternatively, the final concentration of yeast extract can be in the range 0.5% to 1.5%. The broth is then poured aseptically into a sterile plastic Coplin jar. Slides are smeared with the patients' blood, spinal fluid, or synovial fluid. The slides must be −/+, which do not require that fixative be applied. The smears are dried in an aseptic environment before they are placed in the Coplin jar, and the lid tightly closed. Incubation is at 30 degrees C.

### EXAMPLE V

Preparation of Basic Culture Medium for Blood Agar Plates

The Basic MPM Culture Medium broth of EXAMPLE II is modified by adding a total of 1.6% agar. 6% sheep blood is then added as soon as the medium is removed from the autoclave, resulting in “chocolate agar”. At this point, separately autoclaved yeast extract is also added to give a final concentration of 0.5% to 1.5%, preferably 1%. The medium is then poured into plastic Petrie dishes and stored under refrigeration.

### EXAMPLE VI

Culture of Borrelia burgdorferi from Patients with Chronic Lyme Disease

Summary:

Since culture of Borrelia burgdorferi from patients with chronic Lyme disease has been an extraordinarily rare event, clarification of the nature of the illness and proving its etiology as infectious have been difficult. A method for reliably and reproducibly culturing B. burgdorferi from the blood of patients with chronic Lyme disease was therefore sought by making a controlled blood culture trial studying 47 patients with chronic Lyme disease. All had relapsed after long-term oral and intravenous antibiotics. 23 patients with other chronic illness formed the control group. Positive cultures were confirmed by fluorescent antibody immunoelectron microscopy using monoclonal antibody directed against Osp A, and Osp A PCR. 43/47 patients (91%) cultured positive. 23/23 controls (100%) cultured negative. Although persistent infection has been, to date, strongly suggested in chronic Lyme disease by positive PCR and antigen capture, there are major problems with these tests. This new method for culturing B. burgdorferi from patients with chronic Lyme disease certainly defines the nature of the illness and establishes that it is of chronic infectious etiology. This discovery should help to reestablish the gold standard in laboratory diagnosis of Lyme disease.

The study in this EXAMPLE was a multi-center, controlled blood culture trial with an approximately 2:1 ratio of cases to controls. Patients were selected from private practices in areas both hyper-endemic and non-endemic for Lyme disease. All cases had a diagnosis of Lyme disease and had failed or relapsed after extended oral and intravenous antibiotic therapy. The diagnosis of Lyme disease was made primarily on clinical grounds. Although almost all cases had serologic evidence suggestive of infection with B. burgdorferi, few had positive ELISAs and only a little over half met CDC serologic criteria for Western blot positivity. 4/47 (9%) were positive by Lyme ELISA. 3/47 (6%) were equivocal by ELISA. 26/47 (55%/o) were positive by CDC criteria for Lyme Western blot. Of these, 20/26 (77%) were IgM positive, 10/26 (38%) were IgG positive, and 4/26 (15%) were positive for both IgM and IgG.

To participate in the study, all patients had to have had at least 6 consecutive weeks of therapy with an intravenous third-generation cephalosporin and a subsequent relapse. Some patients had had as long as 6 months of intravenous therapy, with the mean being approximately 3 months. Controls resided in non-endemic areas and consisted of patients with chronic illnesses other than Lyme disease.

Two blood samples of 5 ml each were collected in EDTA lavender-top test tubes from each patient and control. From these, seven cultures were processed from each participant. All positive cultures were stained with acridine orange at pH 3.5 4.0 and then confirmed by our laboratory with affinity adsorbed polyclonal fluorescent antibody to B. burgdorferi (02-97-91, Kirkegaard & Perry Laboratories, Gaithersburg, Md., USA).

Further confirmation of positive culture results was accomplished by electron microscopy. Immunoelectron microscopy utilizing monoclonal antibody directed against Osp A (monoclonal antibody no. 181), and plasmid PCR with Osp A primer. The methods employed in these processes have been previously reported (Hulinska et al., 280 Zbl. Bakt. 348-359 (1994); Hulinska et al., 1 Cent Eur. J. Public Health I 81-85 (1993)).

Results:

Of the 47 patients with chronic Lyme disease, 43 (91%) cultured positive for B. burgdorferi, while 23/23 (100%) of the controls cultured negative. Many of the cultures were clearly spirochetes when examined under light microscopy. Immunoelectron microscopy and Osp A PCR confirmation provided additional confirmatory evidence as to the identity of the spirochetes. The slide cultures consistently demonstrated the fastest and most abundant yields. With this technique, placement in the Coplin jar allows for varying gradations of oxygen tension. Sometimes spirochetal growth can be seen after as little as 20 hr. appearing as a band near the upper end of the smear.

Discussion:

We chose to pursue the organism in its cell wall deficient state, i.e., L-forms, as previously reported (Preac-Mursic et al., 24 Infection 218-226 (1996)).

Although L-forms will complex with fluorescent antibody to B. burgdorferi, only as they revert to classic parent forms can the typical spirochetal morphology be seen. There has been a considerable spectrum of cell wall deficiency demonstrated in our laboratory. B. burgdorferi may exist in various forms depending on its environment. In addition to the spirochetal form, we have demonstrated its growth both as amorphous L-forms and rounded giant L-bodies, which have been previously described as cystic forms (Hulinska et al., 280 Zbl. Bakt 348-359 (1994); Brorson & Brorson, 25 Infection 240-246 (1997)). As B. burgdorferi reverts from cell wall deficiency with the rebuilding of its cell wall, classic spirochetal forms can be seen. Most often, in our cultures, B. burgdorferi can be seen in varying stages of reversion, i.e., some L-dependent spirochetal forms within an L-form colony.

The L-form variants, osmotically fragile by nature, require precise conditions to grow in culture. Our medium and methodology are specifically designed for the fostering of cell wall-deficient organisms and their reversion to classic parent forms. We found that 2% yeast extract, instead of 1%, is inhibitory. If the yeast extract is autoclaved with the rest of the medium instead of separately, that too is inhibitory. However, there is one aspect of the B. burgdorferi growth characteristics that we found to be remarkably non-fastidious. The organism can be easily grown throughout a wide range of pH, from 6.8-7.8. This explains the different ratios of NaHCO₃ used in the various types of culture mediums.

It should be noted from this EXAMPLE that currently accepted standards for serologic diagnosis seem to be inadequate. Only a small minority of participants in the study had positive Lyme ELISAs. Under the current recommendations for two-tier testing by the Centers for disease Control/The Association of State and Territorial Public Health Directors (CDC/ASTPHLD), 91% of the patients in the EXAMPLE would have been misdiagnosed as not having Lyme borreliosis.

This EXAMPLE shows that chronic Lyme disease is of chronic infectious etiology, and that even antibiotic treatment well in excess of current recommendations is not necessarily curative. Given the flaws in currently accepted serologic diagnostic criteria, it is likely that Lyme borreliosis is vastly underdiagnosed.

### EXAMPLE VII

Anaerobic Conditions

B. burgdorferi, normally microaerophilic when obtained from tick isolates, becomes progressively more anaerobic as it becomes host adapted, i.e., L-form. As such, we typically age the blood in the actual lavender top tube (see, above) and take advantage of its inherent vacuum. Other methods for anaerobic growth include incubating the culture in culture tubes under anaerobic conditions using a CO₂ pack or other arrangement. This is not to say that B. burgdorferi L-form won't grow if transferred to a normal culture tube and cultured under normal oxygen, they will.

### EXAMPLE VIII

Spirochetes Isolated from 58 Out of 59 Patients with Multiple Sclerosis, a Controlled Series

Summary:

This EXAMPLE concerns the surprising growth of an organism with spirochete morphology from patients with multiple sclerosis (MS). The study population consisted of 59 MS patients. Fifty-four blood samples, four spinal fluids, and one urine were cultured. The medium used is an entirely new sterile preparation that usually permits growth of the spirochetes within 24 hours. Two sets of antibodies to the MS spirochete were produced by vaccination in rabbits with a total of four different spirochetal isolates. Fluorescent antibody assays were then performed with seven isolates from other patients. Six (10%) of the positive cultures were randomly sent out for outside analysis by B. burgdorferi polymerase chain reaction (PCR) and electron microscopy. Fifty-eight out of fifty-nine patients (98%) cultured positive. No spirochetes were cultured from the blood of 36 controls. All indirect fluorescent antibody tests demonstrated positive reactions, suggesting antigenic identity between the isolates used in the procedure. Four out of 6 cultures sent for outside analysis were identified as B. burgdorferi by PCR.

It has long been suspected that multiple sclerosis is an infectious disease, but the link could not be established. We have been able to culture the same spirochete from a series of MS cases. Furthermore, we have produced an antibody from several of the isolates which reacts with the other isolates and proves identity among them. Lastly, 67% of samples sent for outside analysis proved to be B. burgdorferi, suggesting that this organism may be the etiology of MS. Additional findings from this study include intra-erythrocytic growth and high levels of bactericidal antibody in the blood of some healthy individuals.

Introduction.

MS is an enigmatic demyelinating disorder recognized since 1884 (Marie, 12 Prog. Med. (Paris) 287, 305, 349, 365 (1884)). Presently, it is generally believed to be an auto-immune illness. Genetic differences in susceptibility to MS are well known (Kolstad et al., 33 Tissue Antigens 546-549 (1989); Deininger et al., 90 Acto. Neuropathol. 76-79 (1995)) quite likely multi-factorial (Francis et al., 32 Hum-Immunol. 119-124 (1991)), and have been shown to be at least partly due to variation in myelin composition (Chou et al., 49(1-2) J Neuroimmunol. 45-50 (January 1994)). In MS, it is abundantly clear that sensitized T cells progressively destroy the patient's myelin (Ellison, 43 Biomed. & Pharmacother. 327-333 (1989)). It is also evident that its epidemiology suggests that of an infectious disease (Kurtzke, 6 Clinical Microbiology Reviews 382-427 (1993)).

Observations such as case clustering and correlation of disease prevalence with geographic latitude raise suspicion further (Miller et al., 53 Neurosurgery and Psychiatry 903-5 (1990)). A preponderance of MS occurs in individuals who spent some portion of their formative years between the 38^{th} and 52^{nd} latitudes. There is also a well documented increased incidence among doctors and nurses (Shepherd, 54(9) Neurosurgery, & Psychiatry 848-9 (1991)).

Various theories as to modes of transmission of postulated infectious agents have ranged from well water (Warren et al., 10(1) Neuroepidemiology 9-17 (1991)), to household pets (Chan, 1 Lancet 487 (1977)) to mosquitoes (McStreet et al., 15(1) Comparative Immunology, Microbiology, & Infectious Diseases 75-7 (1992)). Another theory postulates that the organism comes from the patient's own oral flora, moving up to penetrate the central nervous system when the hard palate is at a relatively thin growth stage (Gay & Dick, 12 Lancet 75-77 (July 1986)). Yet despite these various theories as to microbial trigger, no conclusive link has been found.

Spirochetes have been associated with MS in three investigative approaches. In the first approach, animal inoculation of MS spinal fluid has resulted in spirochetosis in guinea pigs, hamsters, monkeys, and rabbits. These extensive studies have been summarized by Vincent Marshall (Marshall, 25 Medical Hypotheses 89-92 (1988)). When young rabbits were inoculated, paralysis and death resulted (Kuhn & Steiner, 13 Med. Klin. 1007-9 (1917)). Animal inoculation data were extended by the following careful, intriguing experiment: Human lymphocytes from MS spinal fluid were injected into severe-combined immunodeficient (SCID) mice (Saeki et al., 89 Proc. Natl. Acad. Sci. USA 6157-6161 (1992)). These animals developed paralysis and ataxia. This result has two possible explanations: Either the T cells were able to overcome the species barrier and damaged the murine myelin; or secondly, a pathogen was within the lymphocytes or concentrated on them as the spinal fluid was centrifuged. Interestingly, bacterial L-forms, i.e., cell wall deficient forms, tend to circulate intracellularly in vivo (Mattman, Cell Wall Deficient Forms. (CRC Press, 1993)).

A second approach has been to apply stains to spinal fluid with and without centrifugation, and to stain autopsy tissue. Numerous investigators have found spirochetes in spinal fluids and autopsy nervous tissue. Spirochetes from MS autopsy tissue have reacted with antibody recovered from 50 MS patients' blood (Ibrahim, 214A Abstr. Anat. Rec. 58 (1986); Steiner, 11(4) J Neuropathol. Exp. Neurol. 343 (1952)).

A third approach indicating association of spirochetes with MS has been culture. Compared to inoculation and staining, there have been far fewer attempts. Ichelson had some success (Ichelson, 32 Proc. Penn. Acad. Sci. 49-54 (1958)). Unfortunately, her medium was prone to contamination since it could not be autoclaved. Most unfortunately, several attempts to duplicate Ichelson's work with modifications made to her medium were negative and gave the impression that the spirochetes do not exist (Kurtzke et al., 12 Neurology 915-922 (1962)).

The current report concerns growth of an organism with spirochete morphology from 58 out of 59 MS patients. The fluids yielding positive cultures were as follows: 53 blood samples, four spinal fluids, and one urine. Spirochetes were grown from all active MS cases but were not cultivated from one MS patient in remission. No spirochetes were cultured from the blood of 36 controls.

Methods:

Specimens were cultured by three methods: tubes, slide culture, and agar plates (see, EXAMPLE I above). Tube cultures proved adequate for revealing the spirochetes in most cases. The liquid medium in tubes was the broth which we have designated MPM broth, basic for all of our culturing procedures.

When spinal fluids were cultured by the slide method, a slide with spinal fluid only, a duplicate smeared with a combination of spinal fluid mixed with an equal volume of sheep erythrocytes, and a control consisting of only sheep erythrocytes, were incubated in a Coplin jar.

Two sets of rabbit antibodies were produced. The first set was obtained by using a vaccine composed of formalin-treated blood isolates from three MS patients. The second set resulted from challenging another rabbit with a vaccine composed of a single formalin-treated isolate from the spinal fluid of a fourth patient. A third rabbit, housed in the same animal room, who received no vaccine, was bled at the same intervals as the test rabbits.

Vaccines, always injected with Ribi adjuvant, were administered on days 0 and 28. The first test bleeding of the rabbits was done on day 38. After the initial test bleeding, booster injections were administered at 28 day intervals. Serum was collected 7 days after each booster injection.

The rabbit antisera were used in indirect fluorescent antibody tests. The fluorescein-labeled antibody was anti-rabbit globulin produced by a goat.

Six of the positive cultures were sent out for outside identification by Osp A PCR, nested Osp A PCR, and electron microscopy analysis.

Results.

Spirochetes grew from 53 bloods, 4 spinal fluid samples, and one urine sample, obtained from MS cases. One MS case in remission had negative cultures. Culture of spinal fluid was sometimes negative in the absence of sheep red cells. No spirochetes were cultured from any of the bloods of control patients, nor was there any growth from the control samples of sheep erythrocytes. Growth in broth cultures was usually found by 48 hours. Growth in slide culture was sometimes evident in as little as 20 hours. Growth on the agar plates appeared by two to five days.

Spirochetes from seven MS patients were examined with indirect fluorescent antibody tests. Both sets of rabbit antisera produced from vaccination with a total of four spirochetal isolates demonstrated indirect fluorescent antibody reactions with the spirochetes isolated from the seven other MS patients. Neither antiserum demonstrated reactivity when examined with six cultures of B. burgdorferi isolated from Lyme patients and previously identified by fluorescent antibody to B. burgdorferi. Furthermore, the pre-immunization sera recovered from each rabbit was non-reactive with the cultures from MS or Lyme patients. Similarly, the sera samples from the unvaccinated rabbit did not give fluorescent antibody reactions. Lastly, 4 out of the 6 cultures sent for third party analysis proved to be B. burgdorferi by PCR and electron microscopy.

Our observations associated with this EXAMPLE have shown that spirochetes have surprising characteristics that were not previously recognized. For example, they frequently initiate growth within erythrocytes. Often in clinical studies, we found spirochetes clearly delineated within red cells. Furthermore, spirochetes are not delicate. They can grow in buffered auramine rhodamine stain, which contains approximately 5% phenol. At ambient temperatures, they not only survive, but also slowly multiply, yielding colonies. While refrigerated, we have noted their survival for extended periods of time.

Discussion:

Since spirochetes associated with MS have been repeatedly demonstrated over the years, how does one explain the negative studies in attempts to culture organisms from MS spinal fluids? These negative findings have been reviewed by Kurtzke (Kurtzke, 6 Clinical Microbiology Reviews 382-427 (1993); Kurtzke et al., 12 Neurology 915-922 (1962)). The difference between negative and positive results can be explained at least in part by the fact that Kurtzke and others used pooled human serum. We have discovered that sera from some healthy volunteers markedly inhibits growth of the MS spirochete, suggesting that some people successfully overcome sub-clinical infection with the agent. By chance, any single source of human serum could contain inhibitory antibody, whereas pooled sera would almost assuredly be inhibitory. Furthermore, in no negative study was the medium of Ichelson precisely duplicated.

Some current treatments for MS include relatively toxic substances such as methotrexate (Goodkin et al., 37 Ann. Neurol. 30-40 (1995)), cyclophosphamide (Weiner et al., 43 Neurology 910-918 (1993)), and azathioprine (Yudkin et al., 338 Lancet 1051-1055 (1991)) to suppress “abnormal” immune responses. Interestingly, there are a host of studies using these and other potentially toxic substances for the treatment of MS. Yet for all the suspicion about MS being an infectious disease, there is a conspicuous lack of placebo controlled studies using antibiotics such as tetracycline class which easily cross the blood brain barrier. This EXAMPLE makes the surprising that these trials should take place, given the low toxicity of these drugs and the real potential for benefit.

It is hoped that our findings will help to accurately define the true nature of MS as an infectious disease rather than a primary auto-immune illness. It is likely that MS is caused by infections with B. burgdorferi and other non-burgdorferi, but closely related Borrelia genus, bacteria. Based on our data, double blind placebo controlled trials using antibiotics in the treatment of MS are warranted.

### EXAMPLE IX

Simplified MPM Medium

The following ingredients are mixed:

| | | |
|---|---|---|
| distilled water | 1000 | ml |
| dextrose | 10 | gm |
| sodium chloride | 10 | gm |
| dipotassium phosphate | 4 | gm |
| sodium thioglycollate | 1 | gm |
| purified agar | 1 | gm |
| bacto methylene blue | .004 | gm |
| sucrose | 100 | gm |
| soluble starch | 5 | gm |

This medium is autoclaved for 15 min at 120 degrees C.

Thereafter, yeast extract at a total concentration in the range of from 0.5% to 1.5%, preferably 1%, prepared from the yeast extract solution (Difco 0127), preferably 10%, which has been separately autoclaved for 30 min at 120 degrees C, is added to the solution for improved growth.

We have routinely been using this altered, simplified version of the MPM and it works fine.

### EXAMPLE X

Single Strength Thioglycollate Broth—Negative Results

Difco Thioglycollate Broth (0430) was supplemented with separately autoclaved yeast extract (Difco 0127) added, to give a final concentration in the range of 0.5% to 1.5%, preferably 1% yeast extract. This mixture was tested alternatively in combination with:

1. 6% and 10% rabbit serum

2. 6% and 10% human serum

3. Combination of both rabbit serum and human serum

4. 10% swine serum

Discussion of Experimental Results.

Cultures of blood samples from patients ill with Lyme Disease using the single strength thioglycollate broth taken alone or in combination with any of the additional ingredients recited above all yielded inadequate results and showed little or no positive growth of the organism.

### EXAMPLE XI

Triple Strength Thioglycollate Broth—Negative Results

Difco thioglycollate Broth (0430) was tested both with and without 10% sucrose. Cultures of blood samples from patients ill with Lyme Disease using the triple strength thioglycollate broth taken in combination with any of the additional ingredients recited above all yielded inadequate results and showed no positive growth of the organism.

### EXAMPLE XII

Solid Media—Negative Results

The basic medium for Blood Agar Plate was tested many times without sucrose. Cultures of blood samples from patients ill with Lyme Disease using the basic medium for Blood Agar Plate without sucrose yielded inadequate results and showed no positive growth of the organism.

### EXAMPLE XIII

Veal Infusion Broth—Negative Results

Veal Infusion broth (Difco 0344) was prepared in double strength and tested with and without rabbit serum. Cultures of blood samples from patients ill with Lyme Disease using the double strength veal infusion broth (Difco 0344) either with our without rabbit serum yielded inadequate results and showed no positive growth of the organism.

### EXAMPLE XIV

Thioglycollate Cooked Meat Medium—Negative Results

The following broth is prepared:

cooked meat medium (Difco)

thioglycollate broth (Difco 0236)

The meat granules for one liter of medium are added to a liter of the Thioglycollate Broth. This gives a double strength medium. Cultures of blood samples from patients ill with Lyme Disease using the thioglycollate cooked meat medium taken in combination with any of the additional ingredients recited above all yielded inadequate results and showed no positive growth of the organism.

Summary:

While the experimental results were conducted using a basic cultural medium having the components in the amounts shown, it has been determined that the actual amounts of each of the components of the basic culture composition may vary from about plus or minus 50% and still result in culture media which enable one to effect the growth of cell wall deficient organisms from the blood and other body fluids of patients with chronic illness, especially patients with Lyme Disease and multiple sclerosis. Thioglycollate broth (e.g., from Difco) can be used, in which the concentration of thioglycollate is in the range of between 0.05 weight % (single strength broth) and 0.15 weight % (triple strength broth) of the culture medium, preferably 0.1 weight % (double strength broth). The thioglycollate broth is supplemented with autoclaved yeast extract (e.g., Difco 0127), in which the concentration of yeast extract is in the range of between 0.5 weight % and 1.5 weight % of the culture medium, preferably 1 weight %.

It has been determined that the presence of sucrose is critical to achieving success in the growth of cell wall deficient organisms. The amount of sucrose may vary from a low of about 5% by weight of the base culture medium exemplified to a high of about 50% by weight of the basic culture medium. In one preferred embodiment of the basic culture medium of the invention, sucrose will be present in an amount of from about 5 weight % to about 30%. More preferably, sucrose will be present in an amount of from about 5 weight % to about 20% and most preferably sucrose will be present in an amount of about 10 weight %.

Based upon the foregoing experimental results, it has been determined that the basic culture medium of the invention is a markedly superior vehicle for the growth of cell wall deficient organisms, particularly for the growth of spirochetes, for example, Borrelia, for example Borrelia burgdorferi, obtained from the blood and body fluids of patients with Lyme Disease or multiple sclerosis.

### EXAMPLE XV

Aged Whole Blood Culture Process for Growing Spirochetes from Blood

Scientific Rationale.

Borrelia burgdorferi, as well as other pathogenic and non-pathogenic spirochetes in general, adapt in the mammalian blood environment by loss of spirochete cell wall, resulting in multiple variant forms of the organism (collectively referred to as host adapted or L-forms), including but not limited to the formation of cystic and granular structures. In these altered forms the spirochetes find haven intracellularly and so can better avoid immune destruction.

There are two components to the immune system of animals (in particular, mammals): the cellular and humoral immune response. White blood cells comprise the cellular immune response. The humoral immune response is comprised of non living proteins, antibodies and other proteins, which are formed by certain cells such as white blood cells. If whole blood is left to age at a range of temperature, which includes room temperature, the blood cells begin to die. More specifically to this aged blood culture process, the white blood cells that perform crucial immune system functions become no longer viable. On the other hand, the humoral immune response remains partially intact. Since antibodies and other proteins are nonviable to begin with, they can continue to function in simple aged blood.

However, without live white blood cells to participate in the formation of new antibodies in response to bacterial mutations, the organisms in the blood can adapt somewhat to those antibodies that are present. Another way to overcome the humoral arm of the immune response and thereby allow the bacteria to grow more effectively is to age the blood in the presence of ethylenediaminetetraacetate (EDTA). EDTA, across a broad range of concentrations, inactivates complement, a necessary component to the humoral immune response. For our purposes, we use tri-potassium ethylenediaminetetraacetate (K₃ EDTA) in standard amounts, which are found in the lavender top test tubes routinely used for complete blood counts (1/50-1/10 ml EDTA solution to 2-5 ml blood). The humoral immune response can also be deactivated in a number of other ways. These proteins can be removed by simply pouring off serum, washing blood cells in normal saline and centrifuging down, electrophoresis, introducing blocking antibodies, or a large variety of other ways. However, the idea is the same, to reduce the activity of antibodies and other proteins present in the aged blood so as to allow the bacteria to grow more easily. We have evaluated other methods of deactivating the humoral immune response and they do work to varying degrees However, in our estimation, simply using K₃ EDTA is simpler, less costly, and provides less chance of contamination. EDTA is a slight inhibitor of polymerase chain reaction (PCR), which can hamper analysis of sample results when assessed for growth by PCR. However, given the benefits of using EDTA, we have determined that the benefits outweigh the detriments.

Therefore, because of both the waning cellular and humoral immune responses, the spirochetes are under lessened immune surveillance in an environment, aged blood, which is rich with vital nutrients. The spirochetes, which had been in altered forms due to pressure from the immune response, can now grow in abundance and some may revert back to spiral form. They can now exit intracellular sanctuaries inside red and white blood cells. Although they may regain spiral form more frequently from the blood of patients who have not been treated with antibiotics, even in such patients most grow as L-forms since many blood components, and indeed, even EDTA itself, foster growth preferentially in L-forms.

We have demonstrated that our technique works, not only for Borrelia burgdorferi, but also for other non-human animal (mammalian) chronic spirochetal infections. We have successfully grown and confirmed the growth of non-pathogenic non-Borrelia burgdorferi spirochetes from the blood of healthy beluga whales, horses, and dogs. As such, this technique works for a broad range of chronic spirochetal infections, both pathogenic and non-pathogenic, both from humans, and non-humans.

There are many types of intracellular bacteria. Further, L-forms are not unique to Borrelia burgdorferi. Our method for aged blood is also appropriate for the growth of other chronic blood infections, so long as these organisms do not require the presence of live blood cells for their survival. Examples of such organisms would be bacteria and yeast, but not viruses.

Aside from its advantageous simplicity and greater yields, aged whole blood provides other advantages over other culture methods. Host-adapted Borrelia burgdorferi are very sensitive to pH. Other culture methods require careful monitoring and adjustments of pH. Whole blood, which is very well buffered, does not require such adjustments, and the organisms remain very well protected. Also, since there is no need for manipulation/opening of cultures, there are much less opportunities for outside contamination.

The growth rate of spirochetes from aged blood can be enhanced in the short term by the addition of a 50% sucrose solution. The ratio of 50% sucrose solution to whole blood becomes effective at 1:10 and increases up to 1:2, and then remains constant at 1:1. After addition of sucrose solution, one can appreciate increased spirochetal metabolism of the cultures via acridine orange (AO) staining. AO staining of cystic forms converts from green to red 30 minutes after addition of sucrose, indicating the presence of metabolically active organisms. It is a potentially very quick technique to assess for presence of spirochetes within days because a bright red AO stained cystic form is very easy to see on quick scanning of a slide. However, this increased growth rate is short-lived. The sucrose/whole blood mixture becomes inherently unstable, less able to withstand changes in pH and temperature. Further, because of the necessary adjustments to pH, and the introduction of the sucrose solution itself, despite aseptic technique, the risk of contamination increases. Overall, it is an inferior technique to enhance growth in the long term, but the technique has additive value in the evaluation of short term cultures.

The Preferred Process.

The top of a lavender top CBC tube, with a sterile interior containing tri-potassium ethylenediaminetetraacetate (K₃ EDTA), is sterilized with alcohol. Whole blood is then drawn under aseptic technique into the tube. Almost any kind of aged blood, or blood components, in any tube will work to varying degrees. We use a lavender top K₃ EDTA tube for the convenient combination purpose of inactivating complement and anti-coagulation, but even from aged blood whose complement has not been inactivated and from both clotted blood and serum spirochetal growth can be appreciated after aging, although with decreased yields. The tube of whole blood is then aged at room temperature. Growth can be appreciated over time under extremes of temperature as well. Superior growth is seen between 20 and 37 degrees C. However, growth can be appreciated at any temperature between 0 degrees C and 41 degrees C, but it is extremely slow near freezing and may take many months. Further, growth begins to drop off above 38 degrees C and progressively declines until no growth is seen above 41 degrees C. Assessment for growth can be accomplished by a variety of methods, including but not limited to morphological analysis under bright and dark field, immunofluorescent antibody (IFA) analysis, acridine orange (AO) analysis, antigen capture assays, polymerase chain reaction (PCR), electron microscopy analysis, and electrophoresis of proteins. Growth can usually be appreciated after 6 weeks. Sometimes growth can be appreciated in less time with faster growing strains of B. burgdorferi. For example, growth can occasionally be seen in days. A complete assessment for culture positivity can usually be made after approximately 10 weeks time. Occasionally, a culture that was negative at 10 weeks will turn positive at up to 18 weeks.

When assessing cultures by any direct detection method mentioned above or otherwise, we have a found a means to help concentrate spirochetes, and other organisms as well, from aged blood which can make them easier to detect. This method uses xylene as a concentrating agent. It is not critical to the procedure as a whole but is considered adjunctive. The aged whole blood is diluted with normal saline in a 1:1 ratio in order to prevent the agglutination and coagulation that occurs when undiluted blood is combined with xylene. The range of dilution which is usable is from 1:100 aged blood to normal saline to 5:1 aged blood to normal saline. We have found a ratio of 1:1 to be superior. Xylene, across a broad range of concentrations, i.e., 1:1000 to 10:1 xylene to diluted aged blood can be useful to concentrate the organisms. At lower concentrations of xylene, it is difficult to get enough xylene to be useful, whereas at higher concentrations of xylene, the density of organisms progressively decreases. We use a concentration of xylene to aged blood of 1:10, usually 0.2 ml xylene to 2 ml aged blood, in order to get a dense population of organisms in an accessible amount of xylene.

For Short Term Evaluation.

As described above, whole blood aged for only several days in the presence of K₃ EDTA can be assessed via both IFA and AO, and occasionally, with faster growing strains, growth can be appreciated in these short times. If growth cannot be appreciated, i.e., there are slower growing strains present, after addition of a 50% sucrose solution, one can appreciate increased spirochetal metabolism of the cultures via acridine orange staining. The ratio of 50% sucrose solution to whole blood becomes effective at 1:10, respectively, and increases up to 1:2, and then remains constant at 1:1. Using AO at pH 3.9, staining of cystic forms converts from green to red 30 minutes after addition of sucrose, indicating the presence of metabolically active organisms. It is a potentially very quick technique to assess for presence of spirochetes within days because a bright red acridine orange stained cystic form is very easy to see on quick scanning of a slide.

Adjunctive Uses.

Direct detection tests for B. burgdorferi have been notoriously poor. Since aged blood results in direct spirochetal growth, aged blood has superior results when tested by polymerase chain reaction (PCR) or other direct detection testing. We have had at least 5 separate batches of aged blood tested by PCR analysis. The positivity rates have come in between 75% and 100%, which is superb, considering that for late stage Lyme Disease, blood PCR is about 3% positive.

Although PCR is theoretically an extremely sensitive technique, and in vitro it is been useful for detecting B. burgdorferi (see, above), in vivo PCR uses have many flaws when used to assess presence of B. burgdorferi. As outlined above, as the result of immune pressure, B. burgdorferi alters its form and hides intracellularly, making PCR and other direct detection testing far less sensitive. As aged blood cultures spirochetes, direct detection testing techniques regain their sensitivity. As such, any type of direct detection test can be made more sensitive if the blood is aged, thereby increasing bacterial load. In addition to PCR, examples of such tests would include but not be limited to antigen capture, electrophoresis of proteins, IFA, AO, electron microscopy analysis, and morphological analysis by both bright and dark field.

We have determined that other embodiments of the invention, such as variants on the preferred aged blood technique described above, can also be used successfully. A culture medium used in conjunction with a high blood inoculum can be used, because as the inoculum to medium ratio rises, this approaches the conditions inherent to aged blood. For example, normal saline does not effectively culture B. burgdorferi from patients with Lyme disease and therefore cannot be used. However, by increasing the usually small blood inoculum, then growth can be seen. At ratios of blood inoculum to medium of 1% or greater, some growth can be seen in a variety of media. At ratios of 10% or greater, the conditions mimic aged blood, and appreciable growth is demonstrable.

### EXAMPLE XVI

A Simple and Easily Reproducible Way of Culturing B. burgdorferi from the Blood of Chronic Lyme Disease Patients for Whom Extensive Antibiotic Therapy has Failed

Summary.

The study population was 10 patients with chronic Lyme disease from Lyme borreliosis endemic areas. All had relapsed after long term oral and intravenous antibiotics. The control group was 10 healthy persons from Lyme non-endemic areas. Positive cultures were confirmed by fluorescent antibody and immunoelectron microscopy using monoclonal antibody directed against Osp A, and Osp A PCR. Of the 10 patients, 10 patients (100%) cultured positive. Of the 10 control samples, 1 cultured positive, 3 cultured positive/negative, and 7 cultured clearly negative.

This new, very simple method has clear-cut advantages over other methods. Moreover, this method may help to delineate what the true prevalence of infection with B. burgdorferi is, as well as the asymptomatic to symptomatic infection ratios. This method also may have broad ramifications in clarifying the etiology of a spectrum of other disease states that may be related to infection with B. burgdorferi.

Introduction.

Lyme borreliosis is an enigmatic and controversial multi-system illness caused by infection with B. burgdorferi. Its diagnosis is usually made primarily on clinical grounds, with laboratory data playing merely a supportive role. This emphasis on clinical parameters is largely because laboratory testing for the organism is generally believed to be quite poor. As such, seronegative Lyme borreliosis has been extensively documented (Mouritsen et al., 105(5) Am. J. Clin. Pathol. 647-54 (May 1996); Lawrence et al., 35(2) Eur. Neurol. 113-7 (1995)), as has CSF negative neuroborreliosis Coyle et al., 45(11) Neurology 2010-5 (November 1995); Pfister et al., 39(8) Neurology 1118-20 (August 1989)).

The shortcomings in laboratory diagnosis for this illness fuel controversies in diagnosis, treatment, and definition of cure. There is substantial disagreement over the appropriate treatment for Lyme borreliosis because of the fairly high numbers of patients who remain symptomatic after the frequently prescribed 4 weeks of antibiotic therapy. There is significant human data demonstrating chronic infection with B. burgdorferi as the cause of persistent symptoms in chronic Lyme patients, even despite multiple and extended courses of aggressive antibiotic therapy, as has been demonstrated by both PCR (Nocton et al., 330(4) N. Engl. J. Med. 229-34 (January 1994); Bayer et al., 24(5) Infection 347-53 (September 1996); Priem et al., 57(2) Ann. Rheum. Dis. 118-21 (February 1998); Preac-Mursic et al., 17(6) Infection 355-9 (1989)) and despite the well known difficulties in culturing B. burgdorferi from patients with Lyme borreliosis) also by culture (Pfister et al., 163(2) Infect. Dis. 311-8 (February 1991); Preac-Mursic et al., 13(3) J. Clin. Neuroophthalmol. 155-61 (September 1993)); Preac-Mursic et al., 24(1) Infection.9-16 (1996); Strle et al., 21(2) Infection 83-8 (1993)), in both seropositive and seronegative patients. Even treatment failure in the more easily curable erythema migrans stage Lyme borreliosis has been documented by culture confirmation (Liegner et al., 28(2 Pt. 2) J. Am. Acad. Dermatol. 312-4 (February 1993).

In an effort to help clarify the nature of chronic Lyme borreliosis, we reported on a method for culturing B. burgdorferi from the blood of patients with chronic Lyme borreliosis despite extensive antibiotic therapy (Phillips et al., 26(6) Infection 364-7 (1998); see, EXAMPLE VI). Since that publication, we have to endeavored improve this culture method and to define a very simple and easily reproducible process for culturing B. burgdorferi from the blood.

Patients and Methods.

This EXAMPLE is a blinded controlled blood culture trial. Ten patients carrying a diagnosis of chronic Lyme borreliosis and were chosen from Lyme endemic areas. ten controls were healthy and from areas which are not Lyme endemic. To participate in the EXAMPLE, all patients had to have failed to achieve symptomatic cure with extended antibiotic therapy for Lyme borreliosis. For the purposes of this EXAMPLE, extended antibiotic therapy was defined as at least 4 weeks continuous oral antibiotic therapy with doxycycline plus at least 5 months continuous other oral antibiotics or at least 4 weeks continuous intravenous antibiotic therapy with ceftriaxone plus at least 5 months continuous oral antibiotic therapy. Indeed, most EXAMPLE participants had in excess of 6 months oral antibiotic therapy, in excess of six consecutive weeks of intravenous ceftriaxone therapy. Some participants had been on oral antibiotics for a period of several years and intravenous antibiotics for a period in excess of 3 months. All participants had noted significant improvements in all symptoms with antibiotic therapy, but none had become cured, and all had noted worsening of status off antibiotic therapy.

The diagnosis of Lyme disease was made primarily on clinical grounds with serologic data playing merely a supportive role. For the purposes of this EXAMPLE, a compatible clinical diagnosis for Lyme borreliosis was defined as a multi-system illness with musculoskeletal, neurologic, cardiac, and constitutional complaints in a previously healthy individual from an endemic area. Serology played an important, but secondary role in helping to establish the diagnosis.

In no case was seronegativity by CDC surveillance case definition for the laboratory diagnosis of Lyme borreliosis used to rule out the diagnosis. In no case was lack of correlation with CDC surveillance case definition for the clinical diagnosis of Lyme borreliosis used to rule out the diagnosis. For example, no patients had a history of Bell's palsy, nor frank arthritis, nor atrioventricular block.

The 10 patient samples and 10 healthy control samples were blindly analyzed. Whole blood samples of 5 ml were collected in tri-potassium ethylenediaminetetraacetate (K₃ EDTA) lavender top test tubes from each patient and control. The tops of the tubes were sterilized with isopropyl alcohol prior to phlebotomy. The tubes of whole blood were then left to stand untouched for 4 weeks at 20 degrees C under normal atmospheric conditions and were examined at 2 week intervals until 12 weeks was reached.

Wet preparations were then analyzed under bright and dark field microscopy. All positive cultures were stained with acridine orange at pH 3.9 and then confirmed by our laboratory with affinity adsorbed polyclonal fluorescent antibody to Borrelia burgdorferi (02-97-91, Kirkegaard & Perry Laboratories, Gaithersburg, Md., USA).

The samples can be sent for outside third party evaluation. Confirmation of positive culture results can be accomplished by electron microscopy, immunoelectron microscopy utilizing monoclonal antibody directed against Osp A, and plasmid PCR with Osp A primer. The methodologies employable in these processes have been previously reported (Hulinska et al., 280 Zbl. Bakt 348-59 (1994); Hulinska et al., 1 Cent. Eur. J. Public. Health. 81-5 (1993)).

Results.

Of the 10 patients with chronic Lyme disease, 10 (100%) cultured positive for B. burgdorferi, while 1/10 (10%) of the healthy controls cultured positive. Growth of B. burgdorferi was demonstrated arising from within erythrocytes.

In cultures less than 4 weeks of age, most of the growth was as granular forms appearing to bud from the surface of erythrocytes. As the cultures continued to age, more spheroid L-forms appeared, often as colonies, some with granular and filamentous structures inside, and some with dense cores. As the cultures became still older, some of the faster growing cultures began to attenuate and become less viable, likely from nutrient exhaustion. Immunoelectron microscopy and Osp A PCR confirmation provided additional evidence as to the identity of the organisms.

Discussion.

Microbiologists have been searching for an efficient way to culture B. burgdorferi, and spirochetes in general, from the blood for some time. Our prior method for culturing B. burgdorferi came about as a result of a massive literature search regarding components which have been, to varying degrees, successful for culturing spirochetes and other fastidious organisms. It has the disadvantage of being overly complex and difficult to reproduce.

This EXAMPLE takes the reverse approach. Rather than trying to include multiple growth enhancing ingredients into a complex medium with complex methods, we started with a medium which logically should support the growth of B. burgdorferi, and then removed that which prevents its growth. Simply put, since we have previously cultured B. burgdorferi from the blood with a high yield, then it must follow that the organisms are present in blood in reasonably high levels. As such, logic then dictates that blood itself must contain all the necessary ingredients for growth of the organism.

We made an improvement by expunging that which prevents B. burgdorferi's growth in mammalian blood, a medium to which it should be naturally suited by eons of evolution. In other words, from the blood of the infected patient, we simply withdrew the immune response. For spirochetal growth, it is necessary to neutralize both arms of immune response. By aging the blood at 20 degrees, cellular immunity quickly wanes as the cells involved begin to die. Simple aging does not have an analogous effect on the humoral components of the immune system, but EDTA was used to deactivate compliment and thereby prevent B. burgdorferi destruction (Van Darn et al., 65(4) Infect. Immun. 1228-36 (1997)). Therefore, by aging whole blood with EDTA both arms of the immune response are effectively diminished, which allows growth of the spirochetes from their intracellular sanctuaries.

However, the combination of EDTA and lysozyme, which has been documented to convert B. burgdorferi to L-form (Cluss et al., 64(5) Infect. Immun. 1736-43 (1996)) is present in our method and prevents reversion of B. burgdorferi to spirochete form. As such, parent spirochetal forms are mostly absent, but growth in L-form is effortless and abundant.

B. burgdorferi has been documented intracellularly within multiple cell types, including endothelial cells, fibroblasts, synovial cells, macrophages, and both B and T lymphocytes. However, it has never been documented within erythrocytes. Perhaps this is because microbiologists, too focused on spirochetal forms, have disregarded the possibility of granular forms of spirochetes. Yet, in calves infected with Leptospira interrogans, although there is the clear absence of intra-erythrocytic leptospires, there is the clear presence of intra-erythrocytic granules (Thompson, 96(5) J. Comp. Pathol. 517-27 (September 1996)).

With this simple culture technique, B. burgdorferi grows initially as granular forms. Such forms have been previously reported with B. burgdorferi (MacDonald, Annals New York Academy of Science 468-470 (1998); Kersten et al., 39(5) Antimicrob. Agents Chemother. 1127-33 (May 1995)), as they have with other spirochetes such as Treponema pallidum (Guarner et al., 12(1) Mod. Pathol. 82-7 (January 1999); Delektorskii et al., 4 Vestn. Dermatol. Venerol. 32-6 (1990)), and Leptospirae (Kiktenko et al., 75(10) Bull. Eksp. Biol. Med. 83-6 (October 1973); Alves et al., 44(7) Exp. Toxicol. Pathol. 425-34 (December 1992); Kuiumdzhiev & Mikhailova, 22 Izv. Mikrobiol. Inst. (Sofiia) 135-41 (1971)). As growth begins, small granules, often translucent, appear to be developing within and budding from the surface of erythrocytes.

At the same time, erythrocytes start to sprout spicules and convert to echinocytes with increasing intra-erythrocytic granules. At first, these spicules are non-motile, but over the weeks some of them begin to sprout filamentous structures which then become motile. Soon free swimming highly motile granule and spirochete-like forms are seen. The erythrocytes are progressively lysed. As tempting as it would be to believe that these are all viable organisms, they do not identify as such. Instead, most are consistent with the inert filamentous forms previously described with ecdysis (Rao & Patel, 12(3) Blood Cells 543-53 (1987); Ward et al., 72(3) Am. J. Clin. Pathol. 479-85 (September 1979)) and appear to move by Brownian motion. However, a significant minority is indeed granular forms of B. burgdorferi.

The spontaneous conversion of normal erythrocytes to echinocytes in stored blood has been observed (Laczko et al., 18(4) Haematologia (Budap) 233-48 (1985); Fujisawa & Abiko, 31(9) Arzneimittelforschung 1462-8 (1981)). Although there are multiple theories as to the cause of this phenomenon, there is no clear-cut answer. On the other hand, there are well known causes to some instances of echinocytegenesis (Fetterer & Rhoads, 80(1) Vet. Parasitol. 37-45 (December 1998); Allred et al., 81 J. Cell Sci. 1-16 (March 1986)). It makes good intuitive sense that a variety of insults to erythrocyte membrane integrity could lead to the same characteristic change in shape.

Logically, if an intra-erythrocytic spirochetal infection causes a significant portion of unexplained echinocytes, then patients with compromised immunity, analogously to the declining immune response in EDTA aged blood, should have higher levels of echinocytes. Indeed, it has been demonstrated that patients treated with cyclosporine have increased echinocytes (Ohsako et al., 21(11) Biol. Pharm. Bull. 1236-9 (November 1998)), as do burn patients, uremic dialysis patients, newborns, and cancer patients. Interestingly, decreased immunity occurs in all these groups. Increased echinocytes have also been found in mice genetically prone to the development of cancer. It is well known that heritable deficits in immune surveillance play a major role in such genetically predisposed mice.

Of germane interest, doxorubicin prevents conversion of normal erythrocytes to echinocytes (Thompson et al., 47(11) Cancer Res. 2799-803 (June 1987); Chahwala & Hickman, 45(10) Cancer Res. 4986-9 (October 1985)). At first glance, this may not seem noteworthy. After all, doxorubicin is an antibiotic with only minimal anti-bacterial properties. This begs the question as to what evolutionary pressure would induce Streptomcyes paucitus to produce this antibiotic. As perhaps partial answer to this interesting question, it has been demonstrated that doxorubicin has dramatic anti-bacterial activity against certain L-forms of bacteria without any demonstrable effect against those same parent forms (Gumpert et al., 22(10) Z. Allg. Mikrobiol. 687-92 (1982)). Perhaps doxorubicin prevents formations of echinocytes by interfering with the growth of spirochetal L-forms.

Clearly, several important controversial topics in the field of Lyme borreliosis need to be clarified. First, although seronegative Lyme borreliosis has been well documented, its true prevalence is unknown. In some studies, 71% of patients with chronic Lyme borreliosis were documented to be seronegative by ELISA and therefore negative by CDC case definition criteria (Donta, 25 Suppl 1 Clin Infect Dis :S52-6 (July 1987)). In our previous work, only 9% were seropositive by Lyme ELISA and 6% equivocal by ELISA (Phillips et al., 26(6) Infection 364-7 (1998); see, EXAMPLE VI). In the present EXAMPLE, 100% of the patients did not meet CDC case definition criteria for the diagnosis of Lyme borreliosis.

The statistics of this EXAMPLE documenting the insensitivity of serologic testing for Lyme borreliosis are particularly disturbing when scrutinized in the context of prior studies which dismissed the diagnosis of Lyme borreliosis in large part because of seronegativity. It would seem that these studies are based on assumptions that may not be entirely valid. To more clearly illustrate this point, only 5-7% seropositivity was demonstrated among deer from a Lyme endemic area, despite the mean intensity of infestation with adult I. scapularis on each deer was >180, and that 60% of the adult ticks were infected with B. burgdorferi (Gallivan et al., 34(2) J. Wildl. Dis. 411-4 (April 1998)). Statistically, a very high percentage, perhaps approaching 100%, of these deer should be infected with B. burgdorferi. Yet only 5-7% were seropositive. This leads one to believe that the sensitivity of current serologic testing is absurdly poor for mammals chronically infected with B. burgdorferi. This may be due to a different set of surface proteins expressed in L-forms of B. burgdorferi versus their parent helical forms. Perhaps the 5-7% ELISA positivity is a more realistic approximation of the true sensitivity of Lyme ELISAs for mammals with long term exposure to B. burgdorferi, as would be the case in chronic Lyme borreliosis patients.

Secondly, the data contained in this EXAMPLE would imply that infection with B. burgdorferi, or potentially other Borrelia species, in healthy individuals is quite high. Seroprevalence studies of healthy volunteers have routinely shown surprisingly high seropositivity rates between 9% and 12% (Arocha-Sandoval et al., 35(2) Invest. Clin. 91-104 (June 1994); Jochimsen et al., 89(12) Wis. Med. J. 677-81 (December 1990); Huycke et al., 165(6) J. Infect. Dis. 1133-7 (June 1992); Kaiser et al., 286(4) Zentralbl. Bakteriol. 534-41 (November 1997)). It is extremely unlikely that seropositivity reported in such studies represents false positives, as there is a clear increase in seropositivity in relation to exposure risk to B. burgdorferi (Smith et al., 275(3) Zentralbl. Bakteriol. 382-9 (1991); Nohlmans et al., 135(48) Ned. Tijdschr. Geneeskd. 2288-92 (November 1991); Arzouni et al., 9(3) Eur. J. Epidemiol. 269-73 (May 1993)). Widely recognized that asymptomatic infection with B. burgdorferi is common, most agree that asymptomatic infection greatly predominates over symptomatic infection with this organism (Fahrer et al., 163(2) J. Infect. Dis. 305-10 (February 1991); Zhioua et al., 13(8) Eur. J. Epidemiol. 959-62 (December 1997); Kuiper et al., 12(6) Eur. J. Clin. Microbiol. Infect. Dis.413-8 (June 1993)). Based on the low sensitivity of currently available serologic tests, endemic borrelial infection may be far more common than previously realized. Indeed, if our hypothesis regarding the etiology of echinocytes is correct, their presence in the newborn would imply transplacental infection to be greater than previously thought.

This EXAMPLE may help to prove and disprove a great deal of what is currently accepted as scientific dogma. Endemic borrelial, treponemal, and leptospiral infection has been documented extensively in the medical literature. Our new simple method for culturing B. burgdorferi, by logic, should not be genus or species specific. As such, it can be used to investigate the spectrum of possible spirochetal infections in both healthy individuals and a variety of disease states.

The details of one or more embodiments of the invention are set forth in the accompanying description above. Although any methods and materials similar or equivalent to those described herein can be used in the practice or testing of the invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

The foregoing description has been presented only for the purposes of illustration and is not intended to limit the invention to the precise form disclosed, but by the claims appended hereto.

We claim:

1. An in vitro spirochete culture comprising:
(a) living spirochetes; and
(b) culture medium, wherein the culture medium comprises:
(i) thioglycollate broth, wherein the concentration of thioglycollate is in the range of 0.05 weight % to 0.15 weight % of the culture medium;
(ii) yeast extract, wherein the concentration of yeast extract is in the range of 0.5 weight % to 1.5 weight % of the culture medium; and
(iii) sucrose, wherein the sucrose is present in the medium culture in an amount 5 weight % of the medium culture to 50 weight % of the culture medium
wherein the culture medium has a pH in the range from 6.8-7.8.

2. The in vitro spirochete culture of claim 1, wherein the sucrose is present in an amount of from about 5 weight % to about 30 weight % of the culture medium.

3. The in vitro spirochete culture of claim 1, wherein the sucrose is present in an amount of from about 5 weight % to about 20 weight % of the culture medium.

4. The in vitro spirochete culture of claim 1, wherein the sucrose is present in an amount of about 10 weight % of the culture medium.

5. The in vitro spirochete culture of claim 1, wherein the spirochetes are selected from the group consisting of Borrelia.

6. The in vitro spirochete culture of claim 5, wherein the spirochetes are selected from the group consisting of Borrelia burgdorferi.

7. The in vitro spirochete culture of claim 1, wherein the spirochetes are derived from the blood or other body fluids of humans.

8. The in vitro spirochete culture of claim 7, wherein the humans have a disease selected from the group consisting of Lyme Disease and multiple sclerosis.

9. The in vitro spirochete culture of claim 1, wherein the spirochetes are cell wall deficient organisms.

10. The in vitro spirochete culture of claim 1, wherein the culture medium consists of the following components in the amounts indicated:

| Component | Amount |
|---|---|
| Proteose Peptone | 20 g |
| Beef, infusion from | 1000 g |
| Dextrose | 10 g |
| Sodium Chloride | 10 g |
| Dipotassium Phosphate | 4 g |
| Sodium Thioglycollate | 1 g |
| Agar | 1 g |
| Bacto Methylene Blue | .004 g |
| Sucrose | 100 g |
| Soluble Starch (Difco) | 5 g |
| Water | |

11. The in vitro spirochete culture of claim 1, wherein the culture medium consists of the following components in the amounts indicated:

| Component | Amount | |
|---|---|---|
| water | 1000 | ml |
| sucrose | 10 | gm |
| sodium chloride | 10 | gm |
| dipotassium phosphate | 4 | gm |
| sodium thioglycollate | 1 | gm |
| purified agar | 1 | gm |
| bacto methylene blue | .004 | gm |
| sucrose | 100 | gm |
| soluble starch | 5 | gm |
| yeast extract | 10 | g |

12. A method for identifying the presence of a spirochete in a human, comprising:
(1) obtaining a sample of blood or other body fluid from a human;
(2) culturing the sample in a culture medium, wherein the culture medium comprises:
(a) thioglycollate broth, wherein the concentration of thioglycollate is in the range of 0.05 weight % to 0.15 weight % of the culture medium;
(b) yeast extract, wherein the concentration of yeast extract is in the range of 0.5 weight % to 1.5 weight % of the culture medium; and
(c) sucrose, wherein the sucrose is present in the medium culture in an amount in the range of 5 weight % of the medium culture to 50 weight % of the culture medium; and
(3) identifying the presence of the spirochete in the culture, wherein the identification of the spirochete in the culture identifies the presence of the spirochete in the human.

13. The method of claim 12, wherein the spirochete is selected from the group consisting of Borrelia.

14. The method of claim 13, wherein the spirochete is selected from the group consisting of Borrelia burgdorferi.

15. The method of claim 12, wherein the identifying of the presence of the spirochete in the culture is by fluorescent antibody immunoelectron microscopy using a monoclonal antibody that binds to Osp A.

16. The method of claim 12, wherein the identifying of the presence of the spirochete in the culture is by a polymerase chain reaction using primers that amplify an Osp A polynucleotide.

17. The method of claim 16, wherein the human is suspected of having a disease selected from the group consisting of chronic Lyme Disease and multiple sclerosis.

18. A method for identifying the presence of spirochetes in a mammal, comprising:
(1) obtaining a sample of blood from a mammal in a container, such that the obtained blood sample contains ethylenediaminetetraacetate (EDTA), in a concentration sufficient to inactivate complement in the blood sample;
(2) aging the blood sample, wherein the temperature is in the range of 0 degrees C to 41 degrees C, and
(3) assessing the growth of spirochetes in the blood sample.

19. The method of claim 18, wherein the temperature is in the range of 20 degrees C to 37 degrees C.

20. The method of claim 18, wherein the temperature is room temperature.

21. The method of claim 18, wherein the assessing the growth of spirochetes in the blood sample is by a method selected from the group consisting of morphological analysis by microscopy, immunofluorescent antibody (IFA) analysis, acridine orange (AO) analysis, antigen capture assays, polymerase chain reaction (PCR), electron microscopy analysis, and electrophoresis of proteins.

22. The method of claim 18, further comprising the step of:
prior to the aging of the blood sample, adding sucrose to the blood sample, such that sucrose is present in a concentration in the range of about 5 weight % to 25 weight %.

23. The method of claim 18, wherein the time for aging of the blood sample is in the range of 6 weeks to 18 weeks.

24. The method of claim 18, further comprising the step of:
prior to the assessing the growth of spirochetes, concentrating the spirochetes by:
(i) diluting the blood sample with normal saline in a ratio in the range of 1:100 aged blood to normal saline to 5:1 aged blood to normal saline;
(ii) adding xylene to the diluted blood sample, wherein the ratio of added xylene to diluted blood sample is in the range of 1:1000 to 10:1; and
(iii) collecting the spirochetes by centrifugation.

25. The method of claim 18, wherein the ratio of normal saline to blood is 1:1.

26. The method of claim 18, wherein the ratio of xylene to blood is 1:10.

27. The method of claim 18, wherein the spirochete is selected from the group consisting of Borrelia.

28. The method of claim 27, wherein the spirochete is selected from the group consisting of Borrelia burgdorferi.

29. The method of claim 18, wherein the mammal is selected from the group consisting of human, beluga whale, dog, and horse.

30. The method of claim 18, further comprising the step of:
prior to the aging of the blood sample, diluting the blood sample with normal saline or culture in a ratio in the range of 0:10 aged blood to normal saline or culture medium to 1:10 aged blood to normal saline or culture medium, wherein the culture medium comprises:
(i) thioglycollate broth, wherein the concentration of thioglycollate is in the range of 0.05 weight % to 0.15 weight % of the culture medium;
(ii) yeast extract, wherein the concentration of yeast extract is in the range of 0.5 weight % to 1.5 weight % of the culture medium; and
(iii) sucrose, wherein the sucrose is present in the medium culture in an amount in the range of 5 weight % of the medium culture to 50 weight % of the culture medium
wherein the culture medium has a pH in the range from 6.8-7.8.
